(19) United States
(12) Patent Application Publication
(10) Pub. No.: US 2019/0018003 A1
(43) Pub. Date: Jan. 17, 2019

(54) TUMOR DISCRIMINATION METHOD, DIAGNOSTIC AGENT FOR TUMOR, AND SENSITIZER FOR TUMOR DIAGNOSIS

(71) Applicants: TOTO LTD., KITAKYUSHU-SHI (JP); YOKOHAMA CITY UNIVERSITY, YOKOHAMA-SHI (JP)

(72) Inventors: Koki KANEHIRA, KITAKYUSHU-SHI (JP); Yoshinobu KUBOTA, YOKOHAMA-SHI (JP); Atsuko OTAKE, KITAKYUSHU-SHI (JP); Yukiko YANO, KITAKYUSHU-SHI (JP)

(21) Appl. No.: 16/034,823

(22) Filed: Jul. 13, 2018

(30) Foreign Application Priority Data
Jul. 14, 2017 (JP) 2017-137499

(51) Int. Cl. G01N 33/52; G01N 21/64; C08L 71/08

(52) U.S. Cl. CPC G01N 33/52; G01N 21/6428; G01N 33/57484; C08L 2203/02; C08L 71/08

(57) ABSTRACT

Disclosed is a discrimination method of tumor cells, which can distinguish between tumor cells and normal cells. By administering in vivo a fluorescent dye such as 5-aminolevurinic acid (ALA) and light scattering particles such as titanium oxide particles separately and by irradiating light, there can be obtained fluorescence of such an intensity that makes it possible to distinguish between the tumor cells and the normal cells more definitely than fluorescence obtained with the fluorescent dye alone. Furthermore, the fluorescence emission time is extended by administering in vivo the fluorescent dye and the light scattering particles separately.

### TECHNICAL FIELD

The present invention relates to a method for discriminating between tumor cells and normal cells, and a diagnostic agent and a sensitizer used therefor.

### BACKGROUND ART

In treatment of tumor, a surgical therapy (operation therapy) is the treatment which removes entirely or partially the tumor area. A desired objective of the treatment is to completely remove all of the tumor area appropriately and, for that purpose, it becomes necessary and important to definitely distinguish and discriminate the tumor area, that is, tumor cells which constitute parenchyma of tumor in situ from normal cells. This is because, when the range of removal is not appropriate and a tumor area remains, there is a fear that it may lead to recurrence and metastasis. Further, on the other hand, when even a part to which the tumorr has not spread is removed excessively, there is a fear that possibility of biological function impairment increases, leading to deterioration of quality of life such as postoperative dysfunction and the like.

In recent years, there are performed surgical treatments of tumor by endoscopy because this method imposes little burden on patients, and it has become required that tumor cells or the tumor area are definitely distinguished and discriminated in vivo from normal cells or the normal area.

As a technique to discriminate between the tumor cells and the normal cells, or the tumor area and the normal area, especially in order to discriminate between them in vivo under an endoscope, there has been proposed a technology of imaging, namely, pictorializing and visualizing the tumor cells. For example, WO 91/01727 A discloses a method for detecting and treating the tumor cells by using 5-aminolevulinic acid (ALA). Here, even though ALA by itself has no photosensitivity, it is metabolically activated in the tumor cells into protoporphyrin IX (PpIX) by a series of enzymes of heme biosynthetic pathway, and this is secreted outside the cells and emits light by photoexcitation. By making use of such a property of ALA, the tumor cells are pictorialized and visualized. Furthermore, this method is supposed to be usable for treatment of tumor because singlet oxygen, which is generated by photoexcitation of PpIX, modifies and necrotizes the cells.

Thereafter, there has been proposed such a compound which is specifically modified structurally in the tumor cells and becomes capable of emitting fluorescence by photoexcitation (Nature Communications, 6: 6463 (2015)).

There has been made several proposals such as one to more efficiently pictorialize and visualize the tumor cells by using a fluorescent dye such as ALA and one to further improve the treatment efficiency. For example, JP 2011-1307 A proposes a method for discriminating an accumulation area of PpIX and necrotizing the lesioned tissue by a combination of ALA and light of a plurality of wavelengths.

Furthermore, JP 2009-91345A discloses titanium oxide nanoparticles having a biocompatible polymer binding to the surface thereof, the particles further having ALA binding thereto. These particles, when administered into the body of a cancer patient, reach the cancer tissue efficiently and get accumulated, whereupon irradiation of the affected part with ultrasonic waves and light enables diagnosis and treatment of the cancer. However, it is presupposed that the titanium oxide particles and the ALA disclosed by this patent publication are bonded to each other and are used integrally, and there is no disclosure nor implication to use them separately.

In addition, WO 2012/153493 A discloses a photodynamic therapy agent and a photodynamic diagnostic agent, obtained by combining ALA with particles such as lanthanide particles and the like, which generate up-conversion by infrared range light. The technique disclosed by the patent publication targets a deep-seated cancer.

### SUMMARY OF INVENTION

The present inventors have now found that, by administering in vivo a fluorescent dye and light scattering particles separately, there can be obtained fluorescence of such intensity that makes it possible to distinguish between the tumor cells and the normal cells more definitely than fluorescence obtained with the fluorescent dye alone.

Furthermore, the present inventors have found that the fluorescence emission time is extended by administering in vivo the fluorescent dye and the light scattering particles separately. The present invention is based on these findings.

Accordingly, it is an object of the present invention to provide a method for discriminating between tumor cells and normal cells, or the tumor area and the normal area, and a diagnostic agent and a sensitizer used therefore.

Further, it is also an object of the present invention to provide a discrimination system of tumor cells.

Thus, the method for discriminating the tumor cells according to the present invention is a method for discriminating between tumor cells and normal cells, comprising at least the steps of: (a) taking a fluorescent dye having a tumor selectivity up into the tumor cells; (b) having light scattering particles adsorbed on the surface of the tumor cells and/or uptaken into the tumor cells; and (c) irradiating the tumor cells with light of a wavelength to generate fluorescence in the fluorescent dye at a timing when the fluorescent dye emits fluorescence in the tumor cells.

Furthermore, when the discrimination method is performed in vivo, the discrimination method according to the present invention is such that the step (a) is a step where the fluorescent dye having a tumor selectivity is administered in vivo and the fluorescent dye is uptaken into the tumor cells, and the step (b) is a step where the light scattering particles are administered in vivo and the particles are adsorbed on the surface of the tumor cells and/or uptaken into the tumor cells.

Further, the diagnostic agent according to the present invention is a diagnostic agent to be used for the discrimination method according to the present invention, comprising a fluorescent dye having a tumor selectivity and light scattering particles, wherein the fluorescent dye and the light scattering particles are not bound.

Additionally, the sensitizer according to the present invention is a sensitizer to be used for the discrimination method according to the present invention, comprising light scattering particles.

Furthermore, the discrimination system of tumor cells according to the present invention comprises: (1) a diagnostic agent comprising a fluorescent dye having a tumor selectivity and light scattering particles, wherein the fluorescent dye and the light scattering particles are not bound; (2) a light source which can irradiate light of a wavelength to generate fluorescence in the fluorescent dye to the fluorescent dye uptaken into the tumor cells and the light scattering particles adsorbed on the surface of the tumor cells and/or uptaken into the tumor cells; and (3) an optical device for observing or detecting fluorescence generated in the tumor cells as a result of irradiation by the light source.

According to the present invention, it is possible to enhance light emission of a fluorescent dye in tumor cells and to enhance time of the light emission. As a result, improvement of identifiability of tumor can be achieved.

### BRIEF DESCRIPTION OF DRAWING

FIGS. 1A and 1B show a side view and a plan view, respectively, which simulate a co-culture system pertaining to an embodiment of the present invention.

### DESCRIPTION OF EMBODIMENTS

Discrimination

The method provided by the present invention is a method for discriminating between the tumor cells and the normal cells, and according to one embodiment thereof, the present invention makes it possible, in a body of an animal including human, to discriminate the tumor cells which constitute parenchyma of various tumors and cancers from the normal cells under visible light. Here, the phrase “discriminating between the tumor cells and the normal cells” means distinguishing the tumor cells from the normal cells by some method and determining specific cells to be the tumor cells. Specifically, while a fluorescent dye, details of which will be described later, emits light in the tumor cells, the fluorescent dye does not emit light in the normal cells. Hereby, it becomes possible to determine the cells in an area which emitted light as the tumor cells and to discriminate them from the normal cells. According to the present invention, the fluorescent dye shows enhanced fluorescence and emits light more brightly in comparison to when the fluorescent dye is administered alone. Therefore, the present invention makes it possible, for example, in an endoscopic surgery performed without long incision, to discriminate the tumor cells from the normal cells, which are present in the same visual field and/or in the same area, under a visible light source of the endoscope. And, preferably, it becomes possible to perform a surgical treatment where the area of tumor is removed under a visible light endoscope.

Enhancement of fluorescence in the tumor cells according to the present invention is obtained by using a fluorescent dye and light scattering particles together by separate administration thereof. Compared to an embodiment of JP 2009-91345A where a fluorescent dye is bound to titanium oxide, fluorescence obtained by the present invention is intense selectively in the tumor cells and has a long emission time. Intense emission makes it possible to discriminate even a small tumor from the normal cells and clearly shows a boundary between the tumor area and the normal area. For example, according to a preferable embodiment of the present invention, even a minute tumor of 1 mm or less is visualized to enable reliable removal. Further, it is obvious that the long emission time is advantageous for surgery.

In the present invention, the fluorescent dye and the light scattering particles do no take a configuration where they are bound to each other as described in JP 2009-91345A. Without wishing to be bound by any theory, it is believed that the very bright fluorescence can be observed as a result of the followings: due to presence of a certain physical distance or more between the fluorescent dye and the light scattering particles, irradiation light emitted from a light source, for example, an endoscope or the like, and scattered light generated from the irradiation light by the light scattering particles reach the fluorescent dye effectively to enhance fluorescence intensity; and because the fluorescence light is scattered by the light scattering particles in the side or rear direction thereof, the light emission is enhanced in the direction the irradiation light came from. The present invention is also advantageous in that separate administration of the fluorescent dye and the light scattering particles, as compared to JP 2009-91345 A, makes compounding of a fluorescent dye and light scattering particles unnecessary and increases a degree of freedom in combination of the fluorescent dye and the light scattering particles, making the present invention a highly versatile technology.

Tumor Cells

The tumor cells discriminated by the method according to the present invention are not limited as long as they are of a kind for which the fluorescent dye has selectivity. However, according to one embodiment of the present invention, the method is preferably applied to epithelial tumor cells, non-invasive tumor cells, or tumor cells which constitute parenchyma of carcinoma in situ. The epithelial tumor is, among tumors, one which grows in epithelium and includes the non-invasive tumor and carcinoma in situ formed in a surface region in an early stage of cancer. In such a tumor, a minute cancer of 1 mm or less is a cancer which is difficult to distinguish and tell from a normal area, and the present invention can be applied advantageously to this type of cancer. In addition, the carcinoma in situ is flat and is a cancer which is difficult to distinguish and tell from a normal area, and the present invention can be applied advantageously also in such a cancer when distinguishing the tumor area from the normal area.

According to one embodiment of the present invention, the cancers to which the discrimination method of the present invention is applied include urinary bladder cancer, urothelial carcinoma, colon cancer, gastric cancer, esophageal cancer, cervical cancer, biliary tract cancer, bronchial carcinoma, lung cancer, and brain tumor. These cancers are considered to be objects of surgery under endoscope, and the present invention can be applied advantageously to these cancers.

Fluorescent Dye

The “fluorescent dye having a tumor selectivity” used in the present invention first has “tumor selectivity.” This property means a property of the fluorescent dye to bind to or concentrate in the tumor cells. In addition to this, the term “tumor selectivity” is used in the sense that, while the dye itself does not have a property to bind to or concentrate in the tumor cells, it has a property to become capable of emitting fluorescence selectively in a tumor, that is, for example, even though the original structure of the dye does not have a property to fluoresce, the dye acquires a fluorescent structure as a result of metabolism in the tumor cells.

Further, in the present invention, the term “fluorescent dye” is also used in the sense that it includes not only one having a property to emit fluorescence by itself but also one which acquires a fluorescent structure as a result of some sort of metabolism as described above.

The light which causes emission of the fluorescent dye is not limited as long as it has a wavelength which generates fluorescence. However, according to a preferable embodiment, visible light is preferable because it enables discrimination of tumor cells without using special means of pictorialization and visualization. When ALA is used as the fluorescent dye, it is metabolized and thereby converted to fluorescent PpIX inside the cells, which accumulates especially in the tumor cells. The wavelength of light, which is irradiated to excite this PpIX, includes 380 nm to 420 nm, preferably 400 nm to 410 nm, especially preferably 403 nm to 407 nm, and most preferably 405 nm. Further, even though the light which causes emission of the fluorescent dye is not limited as long as it has a wavelength which generates fluorescence, preferable is a wavelength which can more efficiently enhance fluorescence from the fluorescent dye by means of the light scattering particles.

As an irradiating light source, there can be used publicly known ones. For example, there can be mentioned a violet LED, preferably a flashlight type violet, LED, and laser light such as semiconductor laser and the like. However, more preferable are the violet LED's which make the device compact and are advantageous in terms of cost and portability, including above all a flashlight type violet LED and a violet semiconductor diode.

When ALA is used as the fluorescent dye, the tumor cells can be discriminated by detecting red fluorescence, specifically fluorescence of a wavelength of 610 to 650 nm, preferably 625 to 638 nm in order to detect PpIX which accumulates especially in the tumor cells.

According to a preferable embodiment of the present invention, a specific example of the “fluorescent dye having a tumor selectivity” includes at least one kind selected from the group consisting of 5-amino levulinic acids, porphyrins, hypericins, and enzymatically cleavable dyes. According to a more preferable embodiment, the fluorescent dye having a tumor selectivity includes 5-amino levulinic acids.

In the present invention, the term “5-amino levulinic acids (ALA's)” shall be used in the sense that it includes 5-amino levulinic acid (ALA) or derivatives thereof, or salts of these. Here, as mentioned above, ALA is a publicly known compound and, by itself, absorbs visible light weakly and does not generate fluorescence nor active oxygen by irradiation of light. However, when ALA is administered into the body, it is metabolized into protoporphyrin, which is a photosensitizing substance, to become a fluorescent substance. Accumulation of protoporphyrin, when ALA's are administered, is specific to a lesioned part such as cancer, dysplasia, bacterial or fungal infection site, virus-infected cell, and the like. Also, because ALA's are compounds having high safety, they are preferably used in the present invention.

In the present invention, derivatives of ALA can be represented by the following general formula:

R₁R₂NCH₂COCH₂CH₂COR₃,

wherein R₁ and R₂ are each independently a hydrogen atom, an alkyl group, an acyl group, an alkoxycarbonyl group. an aryl group, or an aralkyl group; and

R₃ is a hydroxy group, an alkoxy group, an acyloxy group, an alkoxycarbonyloxy group. an aryloxy group, an aralkyloxy group, or an amino group.

Accordingly, as specific examples of the ALA derivative, there may be mentioned ALA methyl esters, ALA ethyl esters, ALA propyl esters, ALA butyl esters, ALA pentyl esters, ALA hexyl esters, and the like. Further, as the ALA derivatives, there may be exemplified ALA derivatives having an ester group and an acyl group. As the ALA derivative having an ester group and an acyl group, there may also be mentioned, as preferable examples, those having a combination of a methyl ester group and a formyl group, a methyl ester group and an acetyl group, a methyl ester group and an n-propanoyl group, a methyl ester group and an n-butanoyl group, an ethyl ester group and a formyl group, an ethyl ester group and an acetyl group, an ethyl ester group and an n-propanoyl group, or an ethyl ester group and an n-butanoyl group.

In the present invention, ALA and its derivatives may be in the form of salts, and are preferably pharmaceutically acceptable acid addition salts of inorganic acids or organic acids. As the addition salts of inorganic acids, there may be mentioned, for example, a hydrochloric acid salt, a hydrobromic acid salt, a hydroiodic acid salt, a phosphoric acid salt, a nitric acid salt, and a sulfuric acid salt. As the addition salts of organic acids, there may be mentioned an acetic acid salt, a propionic acid salt, a toluenesulfonic acid salt, a succinic acid salt, an oxalic acid salt, a lactic acid salt, a tartaric acid salt, a glycolic acid salt, a methanesulfonic acid salt, a butyric acid salt, a valeric acid salt, a citric acid salt, a fumaric acid salt, a maleic acid salt, a malic acid salt, and the like. There may also be mentioned metal salts such as a sodium salt, a potassium salt, a calcium salt, and the like; an ammonium salt; alkyl ammonium salts; and the like.

According to a preferable embodiment of the present invention, there may be mentioned, as preferable ALA's; ALA, ALA methyl ester, ALA ethyl ester, ALA propyl ester, ALA butyl ester, and ALA pentyl ester; and hydrochloric acid salts, phosphoric acid salts, and sulfuric acid salts of these.

Additionally, in the present invention, ALA's may form hydrates or solvates, and any one kind may be used alone or two or more kinds may be used in suitable combination. Furthermore, ALA's can be produced by any method including chemical synthesis, production by microorganisms, and production by enzymes.

Light Scattering Particles

The “light scattering particles” used in the present invention mean particles which enhance fluorescence of the above-mentioned fluorescent dye under visible light. Physical phenomena of light related to enhancement of fluorescence include scattering, reflection, interference, refraction, diffraction, and the like of light. Of these, scattering of light includes phenomena such as Rayleigh scattering, Mie scattering, and the like. However, it is thought that the above-mentioned fluorescence of the fluorescent dye is enhanced under visible light, especially, by the Mie scattering. As an important factor related to scattering, there may be mentioned a high refractive index derived from physical properties of a substance which constitutes the light scattering particles. Further, the size of the light scattering particles is preferably equivalent to about 1/10 of the wavelength of light. Visible light generally refers to light in a wavelength range of 400 nm to 700 nm.

According to a preferable embodiment of the present invention, the light scattering particles contain at least one kind of particles selected from the group consisting of titanium oxide, calcium phosphate, hydroxyapatite, alumina, aluminum hydroxide, silica, and polystyrene. More preferable are titanium oxide and polystyrene, which are easy to reach the tumor cells, have a high light scattering effect, and have a low density and a light refraction index. Further, these particles are even more preferably those having a biocompatible polymer binding to the surface thereof. Here, the term “binding to the surface” is used in the sense that at least a portion of a biocompatible polymer is bound to the surface of the particle via a functional group possessed by the biocompatible polymer preferably through multidentate binding, most preferably through bidentate binging, and that allows and includes presence of a biocompatible polymer, which is adsorbed on the surface of the particle not by such binding via a functional group. Alternatively, the term is used in the sense that allows and includes presence of another biocompatible polymer which remains on the surface of the particles by a physical binding (for example, adsorption, entanglement, or the like) to the biocompatible polymer binding to the surface of the particles via a functional group.

According to a more preferable embodiment of the present invention, the light scattering particles are titanium oxide and one having a biocompatible polymer binding to the surface at least partially through multidentate binding.

According to a preferable embodiment of the present invention, the light scattering particles used in the present invention have an average particle size of 60 nm to 400 nm as measured by a dynamic light scattering method with a preferable lower limit of 70 nm and a more preferable lower limit of 80 nm, and with a preferable upper limit of 310 nm and a more preferable upper limit of 200 nm.

Furthermore, details of a biocompatible polymer which is bound at least partially to the surface of the light scattering particles may be the same as the after-mentioned biocompatible polymer which is preferable for titanium oxide. However, according to a preferable embodiment of the present invention, the biocompatible polymer is polyethylene glycol.

According to one preferable embodiment of the present invention, the light scattering particles comprise titanium oxide particles and a biocompatible polymer binding to the surface thereof. According to one embodiment, bonds between the titanium oxide particles and the biocompatible polymer are formed via at least one kind of functional group selected from a carboxyl group, an amino group, a diol group, a salicylic acid group, and a phosphoric acid group. Such binding via a functional group forms a coordinate bond between the biocompatible polymer and titanium oxide and, therefore, the titanium oxide particles can maintain dispersibility despite the fact that they have high catalytic activity. More preferably, this binding includes, from a viewpoint of safety in the body, multidentate binding which secures binding for about 24 to 72 hours after administration into the body. Of the aforementioned functional groups, the functional groups which form multidentate binding are a diol group and a salicylic acid group. The bonds, being multidentate binding, stabilize dispersion of the titanium oxide particles under a physiological condition and suppress isolation of the biocompatible polymer to reduce damage to the normal cells.

According to a preferable embodiment of the present invention, the biocompatible polymer is not particularly limited as long as it can disperse titanium oxide particles in an aqueous solvent. However, as one having a charge, there may be mentioned a biocompatible polymer having anionic property or cationic property, and as one providing dispersibility by hydration without having a charge, there may be mentioned a biocompatible polymer having nonionic property. The biocompatible polymer comprises at least one of these.

According to a preferable embodiment of the present invention, the biocompatible polymer has a weight average molecular weight of 2000 to 100000. The weight average molecular weight of the biocompatible polymer is a value obtained by using size exclusion chromatography. By adjusting the molecular weight in this range, titanium oxide particles can be dispersed to a high degree by action of charge the biocompatible polymer possesses or of hydration even in a near neutral aqueous solvent, where dispersion of titanium oxide particles is considered to be difficult. A more preferable range is 5000 to 100000, even more preferably 5000 to 40000.

According to a preferable embodiment of the present invention, any anionic biocompatible polymer is usable as the biocompatible polymer used in the present invention as long as it can disperse titanium oxide particles in an aqueous solvent. As a biocompatible polymer having a carboxyl group, there may be mentioned, for example, carboxymethyl starch, carboxymethyl dextran, carboxymethyl cellulose, poly-carboxylic acids, and a copolymer with unit of carboxyl groups. Specifically, from a viewpoint of hydrolyzability and solubility of the biocompatible polymer, more suitably used are poly-carboxylic acids such as polyacrylic acid, polymaleic acid, and the like; and copolymers such as copolymers of acrylic acid/maleic acid and acrylic acid/sulfonic acid-based monomer. Even more preferable is polyacrylic acid.

Further, when polyacrylic acid is used as the anionic biocompatible polymer, the weight average molecular weight of the polyacrylic acid is, from a viewpoint of dispersibility, preferably 2000 to 100000, more preferably 5000 to 40000, even more preferably 5000 to 20000. Its structure is not particularly limited, but there may be mentioned a linear structure, a branched structure, a comb-like structure, and the like.

According to a preferable embodiment of the present invention, the biocompatible polymer may be one having amino groups, and specific examples thereof include polyamino acid, polypeptide, polyamines, and a copolymer containing amine units. Further, from a viewpoint of hydrolyzability and solubility of the biocompatible polymer, more suitably used are polyamines such as polyethyleneimine, polyvinylamine, polyallylamine, and the like. Even more preferable is polyethyleneimine.

When polyethyleneimine is used as the cationic biocompatible polymer, the weight average molecular weight of the polyethyleneimine is, from a viewpoint of dispersibility, preferably 2000 to 100000, more preferably 5000 to 40000, even more preferably 5000 to 20000. Its structure is not particularly limited but there may be mentioned a linear structure, a branched structure, a comb-like structure, and the like.

According to another embodiment of the present invention, the biocompatible polymer is a nonionic biocompatible polymer, and there may be mentioned preferably a polymer having a hydroxyl group and/or a polyoxyalkylene group. Examples of such a biocompatible polymer include polyethylene glycol (PEG), polyvinyl alcohol, polyethylene oxide, dextran, or a copolymer containing these, more preferably polyethyelene glycol (PEG) and dextran, even more preferably polyethylene glycol.

When polyethylene glycol is used as the nonionic biocompatible polymer, the weight average molecular weight of the polyethylene glycol is, from a viewpoint of dispersibility, preferably 2000 to 100000, more preferably 5000 to 40000. Its structure is not particularly limited but there may be mentioned a linear structure, a branched structure, a comb-like structure, and the like.

According to a preferable embodiment of the present invention, the titanium oxide particles are anatase-type titanium oxide, rutile-type titanium oxide, or amorphous-type titanium oxide, among which the most preferable is the amorphous-type titanium oxide. According to one embodiment of the present invention, the titanium oxide particles are preferably dispersed in a solvent to be processed into a dispersion form.

According to a preferable embodiment of the present invention, the light scattering particles are further provided with molecules on the surface, the molecules being capable of binding with tumor cells. Here, the molecules capable of binding with the tumor cells are not particularly limited as long as they are molecules which accelerate binding with the tumor cells. However, specific examples include proteins, peptides, nucleic acids, folic acid, or other polymers or low molecules capable of binding with the tumor cells; more preferably proteins, peptides, and nucleic acids; and even more preferably proteins. Antibodies can be used suitably among various proteins. The state of the molecules provided on the surface includes a configuration due to binding of the light scattering particles and molecules capable of binding with the tumor cells, where the binding may be either physical binding or chemical binding. In the chemical binding, when titanium oxide particles are used as the light scattering particles, the bond is formed via at least one kind of functional group selected from a carboxyl group, an amino group, a diol group, a salicylic acid group, and a phosphoric acid group. Such binding via a functional group forms a coordinate bond with titanium oxide and, therefore, the bond between the molecule, which is capable of binding with the tumor cells, and the light scattering particles can be maintained in vivo, although titanium oxide particles have high catalytic activity. More preferably, the binding between the molecule, which is capable of binding with the tumor cells, and the light scattering particles includes, from a viewpoint of safety in the body, multidentate binding which secures binding for about 24 to 72 hours after administration into the body. Of the aforementioned functional groups, ones which form multidentate binding are a diol group and a salicylic acid group. The bonds, being multidentate binding, stabilize dispersion under a physiological condition, suppress isolation of the molecule capable of binding with the tumor cells, and reduces damage to a normal cells.

According to a preferable embodiment of the present invention, such proteins include antibodies against epidermal growth factor receptor or others; growth factors such as epidermal growth factor and the like; glycoproteins such as lectin and the like; and recombinants thereof and the like.

Discrimination Method

Hereinafter, each step of the discrimination method according to the present invention will be described in further detail.

### Step (a)

This step is a step where a fluorescent dye having a tumor selectivity is uptaken into the tumor cells. This step of uptake may specifically be carried out in a state where the fluorescent dye and the tumor cells come into contact. When the fluorescent dye does not have a property to fluoresce by itself but is one which acquires a fluorescent structure as a result of metabolism and the like in the tumor cells, the fluorescent dye should come into contact with the tumor cells in a state where the fluorescent dye can be subjected to such metabolism.

According to another embodiment of the present invention, the discrimination method may be performed not only in vitro but also in vivo and, in the in vivo case, this step (a) shall be a step where the fluorescent dye having a tumor selectivity is administered in vivo and having the fluorescent dye uptaken into the tumor cells. Here, administration in vivo of the fluorescent dye may be either systemic administration or local administration. According to one embodiment of the present invention, the systemic administration includes oral administration, intravenous injection, arterial injection, intraperitoneal administration, infusion, and the like. As the local administration, there is conceived a route of administration where the fluorescent dye is infused in the vicinity of a tumor in each region by means of an endoscope, a catheter, or a syringe, including urinary bladder infusion, enteric infusion, gastric infusion, and the like.

Step (b)

This step is a step where light scattering particles are adsorbed on the tumor cell surface and/or uptaken into the tumor cells. Adsorption or uptake of the light scattering particles are not limited as long as emission of the fluorescent dye is enhanced. For example, the light scattering particles may be adsorbed by contact with or uptaken into permeation through the surface of the tumor cells.

According to another embodiment of the present invention, when the discrimination method is performed in vivo, this step (b) shall be a step where the light scattering particles are administered in vivo to be adsorbed on the surface of the tumor cells and/or to be uptaken into the tumor cells. Here, administration of the light scattering particles may be either systemic administration or local administration but, from a viewpoint of pharmacokinetics, preferable is the local administration. Such local administration is not limited but, according to one embodiment of the present invention, there is conceived a route of administration where the light scattering particles can directly contact the tumor immediately after administration. For example, there can be considered a route of administration where the light scattering particles are infused in the vicinity of a tumor in each region by means of an endoscope, a catheter, or a syringe, including urinary bladder infusion, enteric infusion, gastric infusion, and the like.

In the present invention, the order of steps (a) and (b) does not matter as long as an enhancement effect of emission of the fluorescent dye is obtained. According to one embodiment of the present invention, when the fluorescent dye does not have a property to emit fluorescent light by itself but is one which acquires a fluorescent structure as a result of metabolism and the like in the tumor cells, there are cases where a certain time is needed until the fluorescent structure is obtained. When this time is considered, an order is thought to be efficient where, for example, the step (a) is performed first to have the fluorescent dye uptaken into the tumor cells and, thereafter, the step (b) is performed to have the light scattering particles absorbed on the surface of the tumor cells or uptaken into the tumor cells.

Step (c)

This step is a step where the tumor cells are irradiated with light having a wavelength to generate fluorescence of the fluorescent dye at a timing when the fluorescent dye emits fluorescence in the tumor cells. According to a preferable embodiment of the present invention, the light having a wavelength to generate fluorescence of the fluorescent dye is visible light.

As described above, when the fluorescent dye does not have a property to fluoresce by itself but acquires a fluorescent structure as a result of metabolism and the like in the tumor cells, there are cases where a certain time is needed until the fluorescent structure is obtained. For example, ALA needs 2 hours or more since the time it is administered into the body until it is metabolized to protoporphyrin which is a photosensitizing substance. In the present invention, irradiation of light before attainment of fluorescence is not excluded, but it is efficient to irradiate the tumor cells with light having a wavelength to generate fluorescence of the fluorescent dye at a timing when the fluorescent dye emits fluorescence.

When light is irradiated in this step, the tumor cells emit fluorescence, while the normal cells hardly emit fluorescence. According to the presence or absence, intensity difference, and the position of this fluorescence, the tumor cells and the normal cells are discriminated.

Observation or Detection of Fluorescence

According to one embodiment of the present invention, fluorescence which is generated in the tumor cells or the tumor area by light irradiated from a light source is observed by eyes of man. In this embodiment, the fluorescence is provided as an observation image, and this image is usually obtained through an optical device. Specifically, an image obtained through an endoscope, a colposcope, a digital camera, an optical fluorescence microscope, and the like is visually observed.

In addition, according to another embodiment of the present invention, fluorescence or a specific component thereof is detected through a device, and the detection result may be observed. Such observation of information corresponding to fluorescence or a specific component of fluorescence is preferable because it enables precise recognition not only of presence or absence of fluorescence but also of intensity and the position of generation thereof. As a device for this purpose, there is mentioned an optical device, for example, an optical spectroscopic detector. By applying the optical spectroscopic detector, it becomes possible to detect appropriate fluorescence spectra. By comparing information corresponding to fluorescence or a specific component of fluorescence, for example, fluorescence intensity, at different detection positions in the same visual field or in the same area, the tumor cells and the normal cells, or the tumor area and the normal area can be efficiently discriminated.

Further, according to a preferable embodiment of the present invention, the tumor cells and the normal cells or, the tumor area and the normal area, can be discriminated more advantageously by combining the above-described visually observable images and the information corresponding to fluorescence or a specific component of fluorescence detected through the above-described devices. The tumor cells and the normal cells, or the tumor area and the normal area can be discriminated efficiently by superimposing the information corresponding to fluorescence or a specific component of fluorescence, for example, a relative fluorescence spectrum, with a visually observable image.

According to one embodiment of the present invention, the optical device for observing or detecting fluorescence is more preferably one which observes or detects fluorescence from a direction different from that of light irradiated from a light source. As such an optical device, for example, a combination of optical fiber, lens, and optical spectroscopic detector can be used suitably.

According to one embodiment of the present invention, for example, a surgical treatment to remove the tumor area is carried out in response to the result of the observation or detection. According to a preferable embodiment, the surgical treatment is performed under an endoscope. The endoscope has a visible light source as its lighting, and this visible light can be used as a light source of the emission, which is very advantageous in that it allows precise and efficient removal of the tumorr area.

Diagnostic Agent, Sensitizer, and Discrimination Method of Tumor Cells

As is clear from the foregoing, according to another embodiment of the present invention, there is provided a tumor cell diagnostic agent to be used for the discrimination method according to the present invention, wherein the diagnostic agent comprises a fluorescent dye having a tumor selectivity and light scattering particles wherein the fluorescent dye and the light scattering particles are not bound.

Furthermore, according to another embodiment of the present invention, there is provided a tumor cell diagnostic sensitizer to be used for the discrimination method according to the present invention, wherein the sensitizer comprises the above-described light scattering particles. Here, the light scattering particles are preferably at least one kind selected from the group consisting of titanium oxide, calcium phosphate, hydroxyapatite, alumina, aluminum hydroxide, silica, and polystyrene, and have a biocompatible polymer binding to the surface thereof at least partially through bidentate binding, wherein the biocompatible polymer is more preferably polyethylene glycol.

Further, according to another embodiment of the present invention, there is provided a discrimination system of tumor cells, wherein the discrimination system comprises the following (1) to (3): (1) a diagnostic agent comprising a fluorescent dye having a tumor selectivity and light scattering particles, wherein the fluorescent dye and the light scattering particles are not bound; (2) a light source which can irradiate the fluorescent dye uptaken into the tumor cells and the light scattering particles adsorbed on the surface of the tumor cells and/or uptaken into the tumor cells with light of a wavelength which generates fluorescence in the fluorescent dye; and (3) an optical device for observing or detecting fluorescence generated in the tumor cells as a result of irradiation with the light source.

### EXAMPLES

The present invention will be described in more detail with reference to the following examples, but the present invention is not limited by these examples.

### Example 1

### Preparation of Light Scattering Particles (1)

Titanium tetra-ethoxide was added to an acetonitrile/ethanol solution to prepare a 0.1 mmol/l titanium tetra-ethoxide solution. Into this solution was mixed ethanol and 0.1 mmol/l aqueous ammonia, and the mixture was stirred at room temperature for 60 minutes to carry out hydrolysis sufficiently. Hereat, four kinds of the amount of aqueous ammonia were adjusted in a range of 0.01 to 1 v/v % of the solution depending on target average particle size. After hydrolysis, the reaction mixture was stirred at 80° C. for 3 hours or more under reflux. Further, in order to obtain a solid content thus prepared, the mixture was centrifuged at 20000 g for 10 minutes and the concentration was adjusted by methanol to a solid content of about 20 w/v % to obtain dispersions of 4 kinds of light scattering particles (1) (i) to (iv).

The 4 kinds of light scattering particles (1) (i) to (iv) were each adjusted to a solid content of 0.005 w/v % using ultrapure water and average particle sizes were measured by a dynamic light scattering method and cumulant analysis by using a dynamic light scattering measuring device (manufactured by Spectris Co., Ltd., Zetasizer NANO ZS). As a result, the average particles sizes were respectively: (i) 86.5 nm, (ii) 133.5 nm, (iii) 204.4 nm, and (iv) 330 nm. Also, PDI's (polydispersity index) were respectively (i) 0.047, (ii) 0.017, (iii) 0.017, and (iv) 0.017.

### Example 2

### Preparation of Light Scattering Particles (2) Having Dispersant Binding to Surface Thereof

To 1 g of a copolymer (average molecular weight: 33659; produced by NOF CORPORATION) of polyoxyethylene-monoallyl-monomethyl ether and maleic acid anhydride, as PEG, was added 5 ml of ultrapure water and, after hydrolysis, the solution obtained and 1-ethyl-3-(3-dimethylaminopropyl)carbodiimide hydrochloride (produced by Dojindo Molecular Technologies, Inc.) were mixed and prepared, and concentrations were adjusted by using ultrapure water to 50 mg/l and 50 mmol/l, respectively. To the solution prepared was added 4-aminosalicylic acid (FUJIFILM Wako Pure Chemical Corporation) so that its concentration became 0.1 M, and the mixture was reacted by shaking and stirring for 24 hours at room temperature. After the reaction, the solution obtained was transferred to a Spectra/Por CE dialysis tubing (cut off molecular weight: 3500, Spectrum Laboratories, Inc.), and dialysis was performed for 24 hours at room temperature. After dialysis, the solution was freeze-dried, and to powder obtained was added dimethyl formamide (DMF: FUJIFILM Wako Pure Chemical Corporation) so that a solid content became 25 mg/ml. The mixture was mixed to obtain a solution of PEG bound with 4-aminosalicylic acid.

Subsequently, 20 ml of reaction solutions were prepared by adjusting the solution of PEG bound with 4-aminosalicylic acid to a final concentration of 1.5 mg/ml with DMF and the particles (1) (i) to (iv) having different average particle sizes obtained in Example 1 to a solid content of 0.5 w/v % as the final concentration. These reaction solutions were heated at 130° C. for 16 hours. After completion of the reaction, the solutions were cooled until the temperature of the reaction vessel became 50° C. or less, and DMF was removed by an evaporator to complete dryness. Hereafter, operation was performed in a clean bench aseptically, and a light scattering particle solution obtained by adding sterilized ultrapure water to the powder and mixing was transferred to a sterilized 50 ml tube. Thereafter, centrifugation was performed at 20000 g for 10 minutes and 90 v/v % of the solution was removed and replaced with sterilized ultrapure water. This operation was repeated 8 times. Ultimately, final concentration was adjusted to 1.0 w/v % to obtain 10 ml of solution. Thus, there were prepared light scattering particles (2) (i) to (iv) having a biocompatible polymer binding thereto.

The light scattering particles (2) (i) to (iv) having a biocompatible polymer binding thereto were subjected to measurement of average particle size by cumulant analysis in the same manner as in Example 1. As a result, the average particle sizes were respectively: light scattering particles (2) (i) (prepared from particles 1 (i) having an average particle size of 86.5 nm) 83.3 nm; light scattering particles (2) (ii) (prepared from particles 1 (ii) having an average particle size of 133.5 nm) 129 nm; light scattering particles (2) (iii) (prepared from particles 1 (iii) having an average particle size of 204.4 nm) 198.6 nm; and light scattering particles (2) (iv) (prepared from particles 1 (iv) having an average particle size of 330 nm) 304.4 nm. Also, PDI's (polydispersity index) were respectively (i) 0.043, (ii) 0.019, (iii) 0.008, and (iv) 0.019.

### Example 3

### Preparation of Tumor Cells and Immortalized Normal Cells

All cell cultures were performed by using a CO₂ incubator (Panasonic, MCO-230AICUV-PJ) at 3TC under 5 v/v % CO₂ and humidified conditions. Further, all centrifugations were performed by using a desk-top centrifuge (KOKUSAN H-36) under conditions of 220×g and 6 minutes.

(1) Preparation of Tumor Cells (T24, Human Urinary Bladder Cancer Cell Line)

T24 cells (T24, JCRB0711) were prepared. This cell line was subcultured in an MEM medium (MEM, GlutaMAX™ supplement (Thermo Fisher Scientific), 10 v/v % FBS (Thermo Fischer Scientific)). The cultured cells which reached a logarithmic growth phase 3 days or 4 days later were peeled off with Trypsin/EDTA (Thermo Fisher Scientific) and, after the reaction was terminated in an MEM medium, centrifuged. The cell pellet obtained was suspended in an MEM medium. The cell suspension was subjected to measurement of cell density, inoculated onto a 6-well plate at a density of 3.6×10⁴ cells/2 ml/well, and cultured for 3 days.

(2) Preparation of Tumor Cells (UM-UC-3, Human Urinary Bladder Cancer Cell Line)

UM-UC-3 cells (UMUC3, ATCC CRL-1749) were prepared. This cell line was subcultured in an E-MEM medium (ATCC-formulated Eagle's Minimum Essential Medium (ATCC), 10 v/v % FBS (Life Technologies)). The cultured cells which reached a logarithmic growth phase 3 days or 4 days later were peeled off with Trypsin/EDTA (Thermo Fisher Scientific) and, after the reaction was terminated in an E-MEM medium, centrifuged. The cell pellet obtained was suspended in an E-MEM medium. The cell suspension was subjected to measurement of cell density, inoculated onto a 6-well plate at a density of 3.6×10⁴ cells/2 ml/well, and cultured for 3 days.

(3) Preparation of Tumor Cells (DLD-1, Human Colon Cancer Cell Line)

DLD-1 cells (DLD-1, JCRB9094) were prepared. This cell line was subcultured in an RPMI-1640 medium (RPMI-1640 medium (Life Technologies), 10 v/v % FBS (Life Technologies)). The cultured cells which reached a logarithmic growth phase 3 days or 4 days later were peeled off with Trypsin/EDTA (Thermo Fisher Scientific) and, after the reaction was terminated in an RPMI-1640 medium, centrifuged. The cell pellet obtained was suspended in an RPMI-1640 medium. The cell suspension was subjected to measurement of cell density, inoculated onto a 6-well plate at a density of 2.9×10⁵ cells/2 ml/well, and cultured for 1 day.

(4) Preparation of Immortalized Normal Cells (WI-38, VA13 sub 2 RA, Human Embryonal Lung Cell Line)

Immortalized cell line WI-38 cells (WI-38 VA13 sub 2 RA, JCRB9057) of normal diploid fibroblast cell line WI-38 were prepared. This cell line was subcultured in an MEM medium (MEM, GlutaMAX™ supplement (Thermo Fisher Scientific), 10 v/v % FBS (Thermo Fischer Scientific)). The cultured cells which reached a logarithmic growth phase 3 days or 4 days later were peeled off with Trypsin/EDTA (Thermo Fisher Scientific) and, after the reaction was terminated in an MEM medium, centrifuged. The cell pellet obtained was suspended in an MEM medium. The cell suspension was subjected to measurement of cell density, inoculated onto a 6-well plate at a density of 5.0×10⁴ cells/2 ml/well, and cultured for 3 days.

### Example 4

### Fluorescence Enhancement Effect in T24 Urinary Bladder Cancer Cells by Separate Administration of Fluorescent Dye and Particles (2) (ii)

An aqueous ALA solution (50 mmol/l) was mixed into an MEM medium (MEM, GlutaMAX™ supplement (Thermo Fisher Scientific)) to form a 2 mmol/l ALA solution. Further, an aqueous solution of light scattering particles (2) (ii) was mixed into an MEM medium to form a 0.001 w/v % light scattering particle solution.

Evaluation was performed as follows. The medium of the T24 cells on a 6-well plate, obtained in Example 3, was removed by an aspirator, and thereto was added 2 ml of PBS (−) (Thermo Fisher Scientific). Once again, PBS (−) was removed by an aspirator, thereto was added 2 ml of an ALA solution, and the system was cultured for 2 hours. Then, the ALA solution on the 6-well plate was removed by an aspirator, thereto was added 2 ml of a light scattering particle solution, and the system was cultured for 2 hours. Further, the particle solution on the 6-well plate was removed by an aspirator, the residue was washed with HBSS (−) (Thermo Fisher Scientific), HBSS (−) was removed gain by an aspirator, to the residue was added 2 ml of HBSS (−), and the solution was used for observation and detection of fluorescence.

Observation and detection of fluorescence was performed by using an inverted fluorescence microscope (ECLIPSE Ti-E, Nikon). By using a halogen light source lamp and a 450 nm dichroic mirror, excitation light was irradiated through a 410 nm band pass filter of a 25 nm half-band width, and fluorescence was passed through a 600 nm long-pass filter. There were used an eyepiece of 10 times magnification and an objective lens of 20 times magnification and numerical aperture of 0.75. The irradiation aperture, exposure time, and gain were set to ND=8, 400 ms, and 14.0×, respectively. Fluorescent images were acquired by a cooling CCD color camera (DS-Fi3, Nikon) as digital images. By using an image analysis device IS-Elements AR ver.4.60 (Nikon), fluorescence was detected by subtracting luminance corresponding to dark noise from the image acquired, thereafter obtaining average luminance of all pixels where fluorescence is acquired, and calculating a fluorescence intensity. Also, the image acquired was observed visually.

Relative fluorescence intensity was calculated by using the fluorescence intensities acquired above according to the following formula:

[relative fluorescence intensity]=[fluorescence intensity under each condition]/[fluorescence intensity immediately after irradiation of observation light in the case of administration of fluorescent dye only].

The results were as shown in Table 1.

TABLE 1

| | | Immediately | 50 seconds | 150 seconds |
|---|---|---|---|---|
| | | after | after | after |
| | | observation | observation | observation |
| | | light | light | light |
| | | irradiation | irradiation | irradiation |
| Relative | administration | 1.0 | 0.4 | 0.1 |
| fluorescence | of fluorescent | | | |
| intensity | dye only | | | |
| | separate | 3.4 | 1.6 | 0.5 |
| | administration | | | |
| | of fluorescent | | | |
| | dye and light | | | |
| | scattering | | | |
| | particles (2) (ii) | | | |

As is clear from Table 1, in the case of separate administration of the fluorescent dye and the light scattering particles (2) (ii), a very high relative fluorescence intensity was obtained immediately after irradiation of observation light than in the case of administration of the fluorescent dye only. Further, even at 150 seconds after irradiation of observation light, a high relative fluorescence intensity was observed similarly in the case of separate administration of the fluorescence dye and the light scattering particles (2) (ii). Such high relative fluorescence intensity is thought to be a result of intensification of scattering of visible light by the light scattering particles (2) (ii) administered separately from the fluorescent dye, and enhancement thereby of fluorescence emitted from the fluorescent dye. Further, from a result of visual observation also, it could be confirmed that brightness could be maintained clearly for a longer time when the light scattering particles were present.

From the above, it became clear that, by using the fluorescent dye and the light scattering particles (2) (ii) by separate administration in the present invention, fluorescence is enhanced than in the conventional case of administration of the fluorescent dye only, and the T24 urinary bladder cancer cells can be discriminated more brightly for a longer time.

### Example 5

### Fluorescence Enhancement Effect in UM-UC-3 Urinary Bladder Cancer Cells by Separate Administration of Fluorescent Dye and Light Scattering Particles (2) (ii)

An aqueous ALA solution (50 mmol/l) was mixed into an E-MEM medium (ATCC-formulated Eagle's Minimum Essential Medium (ATCC)) to form a 2 mmol/l ALA solution. Further, an aqueous solution of light scattering particles (2) (ii) was mixed into an E-MEM medium to form a 0.001 w/v % light scattering particle solution.

Evaluation was performed as follows. The medium of the UM-UC-3 cells on a 6-well plate, obtained in Example 3, was removed by an aspirator, and evaluation thereof was performed under conditions where PBS (−) and HBSS (−) in Example 4 were changed to the E-MEM medium and the MEM medium (no glutamine and no phenol red (Thermo Fisher Scientific), respectively, and the solution was used for detection of fluorescence.

Detection of fluorescence was performed in the same manner as in Example 4 by using an inverted fluorescence microscope (ECLIPSE Ti-E, Nikon). A fluorescence intensity was calculated by subtracting luminance corresponding to dark noise from the image acquired and thereafter obtaining average luminance of all pixels where fluorescence was acquired. A relative fluorescence intensity was calculated by using the fluorescence intensities acquired above according to the following formula:

[relative fluorescence intensity]=[fluorescence intensity per unit area under each condition]/[fluorescence intensity per unit area in the case of administration of fluorescent dye only].

The results were as shown in Table 2.

TABLE 2

| | | Immediately after |
|---|---|---|
| | | observation light |
| | | irradiation |
| Relative | administration of | 1.0 |
| fluorescence | fluorescent dye | |
| intensity | only | |
| | separate | 3.5 |
| | administration of | |
| | fluorescent dye | |
| | and light | |
| | scattering | |
| | particles (2) (ii) | |

As is clear from Table 2, in the case of separate administration of the fluorescent dye and the light scattering particles (2) (ii), a very high relative fluorescence intensity was obtained immediately after irradiation of observation light than in the case of administration of the fluorescent dye only. This is thought to be a result of intensification of scattering of visible light by the light scattering particles (2) (ii) administered separately, and enhancement thereby of fluorescence emitted from the fluorescent dye. From the above, it became clear that, by using the fluorescent dye and the light scattering particles (2) (ii) by separate administration, fluorescence is more enhanced than in the conventional case of administration of the fluorescent dye only, and the UM-UC-3 urinary bladder cancer cells can be discriminated more brightly.

### Example 6

### Fluorescence Enhancement Effect in DLD-1 Colon Cancer Cells by Separate Administration of Fluorescent Dye and Light Scattering Particles (2) (ii)

An aqueous ALA solution (50 mmol/l) was mixed into an RPMI-1640 medium (Life Technologies) to form a 2 mmol/l ALA solution. Further, an aqueous solution of light scattering particles (2) (ii) was mixed into an RPMI-1640 medium to form a 0.001 w/v % light scattering particle solution.

Evaluation was performed as follows. The medium of the DLD-1 cells on a 6-well plate, obtained in Example 3, was removed by an aspirator, and evaluation thereof was performed under conditions where PBS (−) and HBSS (−) in Example 4 were changed to RPMI-1640 medium and RPMI-1640 medium (no glutamine and no phenol red (Thermo Fisher Scientific), respectively, and the solution was used for detection of fluorescence.

Detection of fluorescence was performed in the same manner as in Example 5 by using an inverted fluorescence microscope and, from the acquired images, fluorescence intensities were obtained and relative fluorescence intensities were calculated.

The results were as shown in Table 3.

TABLE 3

| | | Immediately |
|---|---|---|
| | | after observation |
| | | light irradiation |
| Relative | administration of | 1.0 |
| fluorescence | fluorescent dye only | |
| intensity | separate | 1.5 |
| | administration of | |
| | fluorescent dye and | |
| | light scattering | |
| | particles (2) (ii) | |

As is clear from Table 3, in the case of separate administration of the fluorescent dye and the light scattering particles (2) (ii), a very high relative fluorescence intensity was obtained immediately after irradiation of observation light than in the case of administration of the fluorescent dye only. This is thought to be a result of intensification of scattering of visible light by the light scattering particles (2) (ii) administered separately, and enhancement thereby of fluorescence emitted from the fluorescent dye. From the above, it became clear that, by using the fluorescent dye and the light scattering particles (2) (ii) by separate administration in the present invention, fluorescence is more enhanced than in the conventional case of administration of the fluorescent dye only, and the DLD-1 colon cancer cells can be discriminated more brightly.

### Example 7

### Fluorescence Enhancement Effect in T24 Urinary Bladder Cancer Cells by Separate Administration of Fluorescent Dye and Light Scattering Particles (2) (iv)

An aqueous ALA solution (50 mmol/l) was mixed into an MEM medium (MEM, GlutaMAX™ supplement (Thermo Fisher Scientific)) to form a 2 mmol/l ALA solution. Further, an aqueous solution of light scattering particles (2) (iv) was mixed into an MEM medium to form light scattering particle solutions of 0.01, 0.1, and 0.5 w/v %.

The medium of the T24 cells on a well plate, obtained in Example 3, was removed by an aspirator, and evaluation thereof was performed under the same conditions as in Example 4, and fluorescence was detected and relative fluorescence intensities were calculated.

The results were as shown in Table 4.

TABLE 4

| | | separate administration | | |
|---|---|---|---|---|
| | | of fluorescent dye and light | | |
| | admin- | scattering particles (2) (iv) | | |
| | istration | Light scattering | Light scattering | Light scattering |
| | of fluo- | particle | particle | particle |
| | rescent | concentration: | concentration: | concentration: |
| | dye only | 0.01 w/v % | 0.1 w/v % | 0.5 w/v % |
| Relative | 1.0 | 1.4 | 1.8 | 0.9 |
| fluo- | | | | |
| rescence | | | | |
| intensity | | | | |

As is clear from Table 4, in the case of separate administration of the fluorescent dye and the light scattering particles (2) (iv), a very high relative fluorescence intensity was obtained, especially when the light scattering particle concentration was 0.1 w/v %, immediately after irradiation of observation light than in the case of administration of the fluorescent dye only. This is thought to be a result of intensification of scattering of visible light by the light scattering particles (2) (iv) administered separately from the fluorescent dye, and enhancement thereby of fluorescence emitted from the fluorescent dye. From the above, it became clear that, by using the fluorescent dye and the light scattering particles (2) (iv) by separate administration in the present invention, fluorescence is more enhanced than in the conventional case of administration of the fluorescent dye only, and the T24 urinary bladder cancer cells can be discriminated more brightly.

### Example 8

### Fluorescence Enhancement Effect in T24 Urinary Bladder Cancer Cells by Simultaneous Administration of Fluorescent Dye and Light Scattering Particles (2) (ii)

An aqueous ALA solution (50 mmol/l) was mixed into an MEM medium (MEM, GlutaMAX™ supplement (Thermo Fisher Scientific)) to form a 4 mmol/l ALA solution. Further, an aqueous solution of light scattering particles (2) (ii) was mixed into an MEM medium to form light scattering particle solutions of 0.001, 0.002, and 0.02 w/v %.

Evaluation was performed as follows. The medium of the T24 cells on a 6-well plate, obtained in Example 3, was removed by an aspirator, and thereto was added 2 ml of PBS (−) (Thermo Fisher Scientific). Once again, PBS (−) was removed by an aspirator, thereto was added 2 ml of a mixed solution obtained by mixing 1 ml of the ALA solution and 1 ml of the light scattering particle solution, and the system was cultured under conditions of 3TC and 5 v/v % CO₂ for 2 hours. Then, the mixed solution of the cells on the 6-well plate was removed by an aspirator, thereto was added 2 ml of HBSS (−) (Thermo Fisher Scientific) to wash the residue, HBSS (−) was removed again by an aspirator, to the residue was added 2 ml of HBSS (−), and the solution was used for detection.

Detection of fluorescence was performed in the same manner as in Example 4 by using an inverted fluorescence microscope and, from the images acquired, fluorescence intensities were obtained and relative fluorescence intensities were calculated.

The results were as shown in Table 5.

TABLE 5

| | admin- | Immediately after observation light irradiation | | |
|---|---|---|---|---|
| | istration | light scattering | light scattering | light scattering |
| | of fluo- | particle | particle | particle |
| | rescent | concentration: | concentration: | concentration: |
| | dye only | 0.0005 w/v % | 0.001 w/v % | 0.01 w/v % |
| Relative | 1.0 | 2.4 | 2.0 | 0.7 |
| fluo- | | | | |
| rescence | | | | |
| intensity | | | | |

As is clear from Table 5, in the case of separate administration of the fluorescent dye and the light scattering particles (2) (ii), a very high relative fluorescence intensity was obtained, especially when the light scattering particle concentration was 0.0005 w/v %, immediately after irradiation of observation light than in the case of administration of the fluorescent dye only. Such high relative fluorescence intensity is thought to be a result of intensification of scattering of visible light by the light scattering particles (2) (ii) administered separately from the fluorescent dye, and enhancement thereby of fluorescence emitted from the fluorescent dye.

From the above, it became clear that, by using the fluorescent dye and the light scattering particles (2) (ii) by simultaneous administration in the present invention, fluorescence is more enhanced than in the conventional case of administration of the fluorescent dye only, and the T24 urinary bladder cancer cells can be discriminated more brightly.

### Example 9

### Fluorescence Enhancement Effect in T24 Urinary Bladder Cancer Cells by Separate Administration of Fluorescent Dye (Hypericin) and Light Scattering Particles (2) (ii)

A solution (100 mmol/l) of hypericin (FUJIFILM Wako Pure Chemical Corporation) in dimethyl sulfoxide (FUJIFILM Wako Pure Chemical Corporation) as a solvent was mixed into an MEM medium (MEM, GlutaMAX™ supplement (Thermo Fisher Scientific)) to form a 1 μmol/l hypericin solution. Further, an aqueous solution of light scattering particles (2) (ii) was mixed into an MEM medium to form a 0.001 w/v % light scattering particle solution.

Evaluation was performed as follows. The medium of the T24 cells on a 6-well plate, obtained in Example 3, was removed by an aspirator, and thereto was added 2 ml of MEM medium (Thermo Fisher Scientific). Once again, the MEM medium was removed by an aspirator, thereto was added 2 ml of hypericin solution, and the system was cultured under conditions of 3TC and 5 v/v % CO₂ for 1 hour. Then, the hypericin solution of the cells on the 6-well plate was removed by an aspirator, to the residue was added 2 ml of the light scattering particle solution, and the system was cultured under conditions of 3TC and 5 v/v % CO₂ for 2 hours. Further, the light scattering particle solution of cells on the 6-well plate was removed by an aspirator, the residue was washed by addition of 2 ml of an MEM medium (no glutamine, no phenol red (Thermo Fisher Scientific)), the MEM medium was removed again by an aspirator, to the residue was added 2 ml of MEM medium, and the solution was used for detection.

Detection of fluorescence was performed in the same manner as in Example 5 by using an inverted fluorescence microscope and, from the images acquired, fluorescence intensities were obtained and relative fluorescence intensities were calculated.

The results were as shown in Table 6.

TABLE 6

| | | Immediately after |
|---|---|---|
| | | observation light |
| | | irradiation |
| Relative | administration of | 1.0 |
| fluorescence | fluorescent dye only | |
| intensity | separate | 10.0 |
| | administration of | |
| | fluorescent dye and | |
| | light scattering | |
| | particles (2) (ii) | |

As is clear from Table 6, in the case of separate administration of the fluorescent dye and the light scattering particles (2) (ii), a very high relative fluorescence intensity was obtained immediately after irradiation of observation light than in the case of administration of hypericin only. This is thought to be a result of intensification of scattering of visible light by the light scattering particles (2) (ii) administered separately, and enhancement thereby of fluorescence emitted from hypericin. From the above, it became clear that, by using hypericin and the light scattering particles (2) (ii) by separate administration in the present invention, fluorescence is more enhanced than in the conventional case of administration of hypericin only, and the T24 urinary bladder cancer cells can be discriminated more brightly.

### Example 10

### Effect of Discriminating between T24 Cancer Cells and WI-38 Immortalized Normal Cells by Separate Administration of Fluorescent Dye and Light Scattering Particles (2) (ii)

An aqueous ALA solution (50 mmol/l) was mixed into an MEM medium (MEM, GlutaMAX™ supplement (Thermo Fisher Scientific)) to form a 2 mmol/l ALA solution. Further, an aqueous solution of light scattering particles (2) (ii) was mixed into an MEM medium to form a 0.001 w/v % light scattering particle solution.

The media of the T24 cells and the WI-38 cells on 6-well plates, obtained in Example 3, were respectively removed by an aspirator, and detection was performed in the same manner as in Example 4, and relative fluorescence intensities were calculated.

The results were as shown in Table 7.

TABLE 7

| | | T24 cell line | WI-38 cell line |
|---|---|---|---|
| Relative | administration of | 1.0 | 0.7 |
| fluorescence | fluorescent dye | | |
| intensity | only | | |
| | separate | 2.6 | 0.8 |
| | administration of | | |
| | fluorescent dye | | |
| | and light | | |
| | scattering | | |
| | particles (2) (ii) | | |

As is clear from Table 7, in the case of separate administration of the fluorescent dye and the light scattering particles (2) (ii), a very high relative fluorescence intensity was obtained in the T24 cell line than in the WI-38 cell line. Further, in the case of separate administration of the fluorescent dye and the light scattering particles (2) (ii), the highest relative fluorescence intensity was obtained under all conditions when T24 cell line was used. Such high relative fluorescence intensity is thought to be a result of intensification of scattering of visible light by the light scattering particles (2) (ii) administered separately from the fluorescent dye, and enhancement thereby of fluorescence emitted from the fluorescent dye. Furthermore, it is thought that the highest relative fluorescence intensity was obtained when T24 cell line was used, due to selectivity of the fluorescent dye for cancer and difference in the property of the particles to be uptaken into the tumor cells and the immortalized normal cells.

From the above, it became clear that, by using the fluorescent dye and the light scattering particles (2) (ii) by separate administration in the present invention, fluorescence is more enhanced than in the conventional case of administration of the fluorescent dye only, and the T24 urinary bladder cancer cells can be discriminated more brightly than the immortalized normal cells.

### Example 11

### Effect of Discriminating between T24 Urinary Bladder Cancer Cells and WI-38 Immortalized Normal Cells, when Present in the Same Visual Field, by Separate Administration of Fluorescent Dye and Light Scattering Particles (2) (ii)

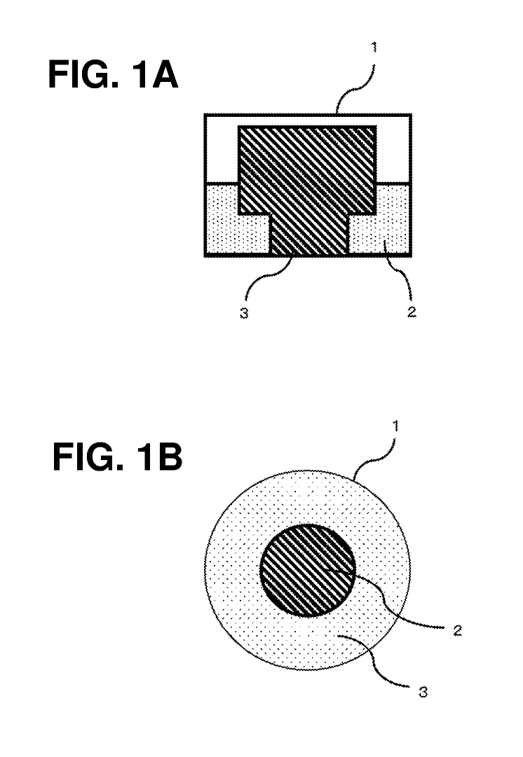

Co-culture pertaining to the present example for having the tumor cells and the immortalized normal cells exist in the same visual field will be described with reference to FIGS. 1A and 1B. For the co-culture, there was used CytoSelect™ 24-Well Cell Co-Culture System (manufactured by COSMO BIO). The T24 cell line was subcultured in an MEM medium (MEM, GlutaMAX™ supplement (Thermo Fisher Scientific), 10 v/v % FBS (Thermo Fisher Scientific)). The cultured cells which reached a logarithmic growth phase 3 days or 4 days later were peeled off with Trypsin/EDTA (Thermo Fisher Scientific) and, after the reaction was terminated in an MEM medium, centrifuged. The cell pellet obtained was suspended in an MEM medium. The cell suspension was subjected to measurement of cell density and inoculated onto a well at a density of 4.4×10⁴ cells/0.225 ml/well, the well having an insert 2 of an 8 mm diameter disposed for preparing a cell-free region therein, and cultured for 2 days to form a monolayer in region 3 around the insert 2.

WI-38 cells were subcultured in an MEM medium. The cultured cells which reached a logarithmic growth phase 3 days or 4 days later were peeled off with Trypsin/EDTA (Thermo Fisher Scientific) and, after the reaction was terminated in an MEM medium, centrifuged. The cell pellet obtained was suspended in an MEM medium. The cell suspension was subjected to measurement of cell density and inoculated onto a well 1, from which the insert 2 had been removed, at a density of 1.0×10⁵ cells/0.5 ml/well and cultured for 1 day.

An aqueous ALA solution (50 mmol/l) was mixed into an MEM medium (MEM, GlutaMAX™ supplement (Thermo Fisher Scientific)) to form a 2 mmol/l ALA solution. Further, an aqueous solution of light scattering particles (2) (iv) was mixed into an MEM medium to form a light scattering particle solution of 0.001 w/v %.

Evaluation was performed as follows. The medium on the co-culture plate was removed by an aspirator, and thereto was added 0.5 ml of MEM medium. Once again, the MEM medium was removed by an aspirator, thereto was added 0.5 ml of an ALA solution, and the system was subjected to incubation for 2 hours. Then, the ALA solution on the co-culture plate was removed by an aspirator, thereto was added 0.5 ml of a light scattering particle solution, and the system was cultured for 2 hours. Further, the particle solution of cells on the co-culture plate was removed by an aspirator, and the residue was washed by adding 0.5 ml of MEM medium (no glutamine, no phenol red (Thermo Fisher Scientific)), the MEM medium was removed again by an aspirator, to the residue was added 0.5 ml of MEM medium, and the solution was used for detection.

Detection of fluorescence was performed in the same manner as in Example 4 by using an inverted fluorescence microscope (ECLIPSE Ti-E, Nikon). Images having a visual field of 3300 μm (width)×2200 μm (length) were obtained, and relative fluorescence intensities were calculated in the same manner as in Example 5.

The results were as shown in Table 8.

TABLE 8

| | WI-38 cell line | T24 cell line |
|---|---|---|
| administration | 1.0 | 1.6 |
| of fluorescent | | |
| dye only | | |
| separate | 1.0 | 2.0 |
| administration | | |
| of fluorescent | | |
| dye and light | | |
| scattering | | |
| particles (2) (ii) | | |

As is clear from Table 8, in the case of separate administration of the fluorescent dye and the light scattering particles (2) (ii), the difference between relative fluorescence intensities of the WI-38 cell line and the T24 cell line. This is thought to be a result of intensification of scattering of visible light by the light scattering particles (2) (ii) administered separately from the fluorescent dye, and enhancement thereby of fluorescence emitted from the fluorescent dye.

From the above, when there are immortalized normal cells and tumor cells on the same plane, it became clear that, by using the fluorescent dye and the light scattering particles (2) (ii) by separate administration in the present invention, fluorescence is enhanced than in the conventional case of administration of the fluorescent dye only, and the difference between fluorescence values of the immortalized normal cells and tumor cells became larger to enable discrimination between them and definite determination of the tumor cell area.

### Example 12

### Fluorescence Enhancement Effect in T24 Urinary Bladder Cancer Cells by Separate Administration of Fluorescent Dye and Silica Particles

An aqueous ALA solution (50 mmol/l) was mixed into an MEM medium (MEM, GlutaMAX™ supplement (Thermo Fisher Scientific)) to form a 2 mmol/l ALA solution. An aqueous solution of silica particles having a particle size of 100 nm (sicastar, Silica Microsphere, Plain, 25 mg/ml, produced by Polyscience) was mixed into the MEM medium to form a 0.01 w/v % silica particle solution.

Evaluation was performed by removing the medium of the T24 cells on a 6-well plate, obtained in Example 3, under conditions where, in Example 4, PBS (−) and HBSS (−) were respectively changed to MEM medium and MEM medium (no glutamine, no phenol red (Thermo Fisher Scientific)), and the solution was used for detection of fluorescence.

Detection was performed in the same manner as in Example 5 by using an inverted fluorescence microscope and, from the images acquired, fluorescence intensities were obtained and relative fluorescence intensities were calculated.

The results were as shown in Table 9.

TABLE 9

| | | Immediately after |
|---|---|---|
| | | observation light |
| | | irradiation |
| Relative | administration of | 1.0 |
| fluorescence | fluorescent dye | |
| intensity | only | |
| | separate | 2.2 |
| | administration of | |
| | fluorescent dye | |
| | and silica particles | |

As is clear from Table 9, a very high relative fluorescence intensity was obtained immediately after irradiation of observation light than in the case of administration of the fluorescent dye only. This is thought to be a result of intensification of scattering of visible light by the silica particles administered separately from the fluorescent dye, and enhancement thereby of fluorescence emitted from the fluorescent dye. From the above, it became clear that, by using the fluorescent dye and the silica particles by separate administration in the present invention, fluorescence is more enhanced than in the conventional case of administration of the fluorescent dye only, and the T24 urinary bladder cancer cells can be discriminated more brightly.

### Example 13

### Fluorescence Enhancement Effect in T24 Urinary Bladder Cancer Cells by Separate Administration of Fluorescent Dye and Polystyrene Particles

An aqueous ALA solution (50 mmol/l) was mixed into an MEM medium (MEM, GlutaMAX™ supplement (Thermo Fisher Scientific) to form a 2 mmol/l ALA solution. Further, as polystyrene particles, an aqueous solution of one having a particle size of 100 nm (Polybead Polystyrene Microspheres 2.5% Solid-Latex, produced by Polyscience) was mixed into the MEM medium to form a 0.01 w/v % polystyrene particle solution.

The medium of T24 cells on a 6-well culture plate, obtained in Example 3, was removed by an aspirator, and evaluation was performed under the same conditions as in Example 12 to perform detection of fluorescence and calculation of relative fluorescence intensities.

The results were as shown in Table 10.

TABLE 10

| | | Immediately after |
|---|---|---|
| | | observation light |
| | | irradiation |
| Relative | administration of | 1.0 |
| fluorescence | fluorescent dye | |
| intensity | only | |
| | separate | 1.4 |
| | administration of | |
| | fluorescent dye | |
| | and polystyrene | |
| | particles | |

As is clear from Table 10, a very high relative fluorescence intensity was obtained immediately after irradiation of observation light than in the case of administration of the fluorescent dye only. This is thought to be a result of intensification of scattering of visible light by the polystyrene particles administered separately, and enhancement thereby of fluorescence emitted from the fluorescent dye. From the above, it became clear that, by using the fluorescent dye and the polystyrene particles by separate administration in the present invention, fluorescence is more enhanced than in the conventional case of administration of the fluorescent dye only, and the T24 urinary bladder cancer cells can be discriminated more brightly.

### Example 14

### Preparation of Light Scattering Particles Having Tumor Cell-Binding Molecule (Antibody) on the Surface and Fluorescence Enhancement Effect Thereof

The light scattering particles (2) (ii) obtained in Example 2 was adjusted so that its solid content became 0.5% with a 50 mM MES buffer solution (pH 5.5). Further, mouse anti-human epidermal growth factor receptor monoclonal antibody (Ab-2 (Clone 225), Thermo Scientific) was mixed therewith so that its final concentration became 50 μg/ml, and the mixture was stirred by shaking at 4° C. for 24 hours to have the antibody adsorbed physically on the surface of light scattering particles (2) (ii). Thereafter, centrifugation was performed at 15000 g for 30 minutes and 90% of the solution was removed and replaced with ultrapure water, and this operation was repeated 3 times. Ultrasonic dispersion under ice cooling was repeated to obtain light scattering particles (2) (ii) having the mouse anti-human epidermal growth factor receptor monoclonal antibody adsorbed on the surface physically. Light scattering particles (2) (v), thus prepared based on the light scattering particles (2) (ii), obtained in Example 2, was adjusted to a solid content of 0.01% by using ultrapure water, and an average particle size was measured in the same manner as in Example 1 by cumulant analysis. As a result, the average particle size of the light scattering particles (2) (v) was 115 nm.

An aqueous ALA solution (50 mmol/l) was mixed into an MEM medium (MEM, GlutaMAX™ supplement (Thermo Fisher Scientific)) to form a 2 mmol/l ALA solution. Further, an aqueous solution (1 w/v %) of light scattering particles (2) (v) was mixed into an MEM medium to form a 0.001 w/v % light scattering particle solution.

The medium of T24 cells on a 6-well culture plate, obtained in Example 3, was removed by an aspirator, and evaluation was performed under the same conditions as in Example 12 to perform detection of fluorescence and calculation of relative fluorescence intensities.

The results were as shown in Table 11.

TABLE 11

| | | Immediately after |
|---|---|---|
| | | observation light |
| | | irradiation |
| Relative | administration of | 1.0 |
| fluorescence | fluorescent dye only | |
| intensity | separate | 1.4 |
| | administration of | |
| | fluorescent dye and | |
| | light scattering | |
| | particles (2) (ii) | |
| | separate | 1.6 |
| | administration of | |
| | fluorescent dye and | |
| | light scattering | |
| | particles (2) (v) | |

As is clear from Table 11, in the case of separate administration of the fluorescent dye and the light scattering particles (2) (v), a very high relative fluorescence intensity was obtained immediately after irradiation of observation light than in the case of administration of the fluorescent dye only and in the case of separate administration of the fluorescent dye and the light scattering particles (2) (ii). This is thought to be a result of intensification of scattering of visible light and enhancement thereby of fluorescence emitted from the fluorescent dye, where the intensification of scattering of visible light is because the light scattering particles (2) (v) administered separately are provided on the surface thereof with molecules capable of binding with tumor cells and, therefore, adsorption of the light scattering particles (2) (v) on the tumor cell surface and/or uptake of the light scattering particles (2) (v) into the tumor cells are accelerated. From the above, it became clear that, by using the fluorescent dye and the particles (2) (v) by separate administration in the present invention, fluorescence is more enhanced than in the conventional case of administration of the fluorescent dye only and the case of separate administration of the fluorescent dye and the particles (2) (ii), and the T24 urinary bladder cancer cells can be discriminated more brightly.

### Example 15

### Preparation of Particles Having Tumor Cell-Binding Molecules (human Epidermal Growth Factor) on the Surface and Fluorescence Enhancement Effect Thereof

The light scattering particles (2) (ii) obtained in Example 2 was adjusted with a 50 mM boric acid buffer solution (pH 5.5) so that the solid content became 0.5%. Further, human Epidermal Growth Factor rhEGF (Animal-derived-free, produced by FUJIFILM Wako Pure Chemical Corporation) was mixed therein so that its final concentration became 50 μg/ml, and the mixture was stirred by shaking at 4° C. for 24 hours to have EGF on the surface of light scattering particles (2) (ii). Thereafter, centrifugation was performed at 15000 g for 30 minutes and 90% of the solution was removed and replaced with ultrapure water. This operation was repeated 3 times. Ultrasonic dispersion under ice cooling was repeated to obtain light scattering particles (2) (ii) having EGF on the surface. Light scattering particles (2) (vi), thus prepared based on the light scattering particles (2) (ii) obtained in Example 2, was adjusted to a solid content of 0.01% by using ultrapure water, and an average particle size was measured in the same manner as in Example 1 by cumulant analysis. As a result, the average particle size of the light scattering particles (2) (vi) was 102 nm.

An aqueous ALA solution (50 mmol/l) was mixed into an MEM medium (MEM, GlutaMAX™ supplement (Thermo Fisher Scientific)) to form a 2 mmol/l ALA solution. Further, an aqueous solution (1 w/v %) of light scattering particles (2) (vi) was mixed into an MEM medium to form a 0.001 w/v % light scattering particle solution.

The medium of T24 cells on a 6-well culture plate, obtained in Example 3, was removed by an aspirator, and evaluation was performed under the same conditions as in Example 12 to perform detection of fluorescence and calculation of relative fluorescence intensities.

The results were as shown in Table 12.

TABLE 12

| | | Immediately after |
|---|---|---|
| | | observation light |
| | | irradiation |
| Relative | administration of | 1.0 |
| fluorescence | fluorescent dye only | |
| intensity | separate | 1.4 |
| | administration of | |
| | fluorescent dye and | |
| | light scattering | |
| | particles (2) (ii) | |
| | separate | 2.2 |
| | administration of | |
| | fluorescent dye and | |
| | light scattering | |
| | particles (2) (vi) | |

As is clear from Table 12, in the case of separate administration of the fluorescent dye and the light scattering particles 2 (vi), a very high relative fluorescence intensity was obtained immediately after irradiation of observation light than in the case of administration of the fluorescent dye only and in the case of separate administration of the fluorescent dye and the light scattering particles (2) (ii). This is thought to be a result of intensification of scattering of visible light and enhancement thereby of fluorescence emitted from the fluorescent dye, where the intensification of scattering of visible light is because the light scattering particles (2) (vi) administered separately are provided on the surface thereof with molecules capable of binding with tumor cells and, therefore, adsorption of the light scattering particles (2) (vi) to the tumor cell surface and/or uptake of the light scattering particles (2) (vi) into the tumor cells are accelerated. From the above, it became clear that, by using the fluorescent dye and the light scattering particles (2) (vi) by separate administration in the present invention, fluorescence is more enhanced than in the conventional case of administration of the fluorescent dye only and in the case of separate administration of the fluorescent dye and the light scattering particles (2) (ii), and the T24 urinary bladder cancer cells can be discriminated more brightly.

### Example 16

### Preparation of Light Scattering Particles (3) Having Biocompatible Polymer Bound to Surface Thereof

Of N-hydroxysuccinimide activated esters of monomethoxy polyethylene glycol as PEG, those having (i) an average molecular weight of 10000 (SUNBRIGHT ME-100GS, produced by NOF CORPORATION), (ii) an average molecular weight of 20000 (SUNBRIGHT ME-200GS, produced by NOF CORPORATION), and (iii) an average molecular weight of 40000 (SUNBRIGHT ME-400GS, produced by NOF CORPORATION) were respectively added to dimethyl formamide (DMF: produced by FUJIFILM Wako Pure Chemical Corporation) and mixed to prepare PEG solutions (i) to (iii), respectively. Further, dopamine hydrochloric acid salt (FUJIFILM Wako Pure Chemical Corporation) was dissolved in DMF to form a dopamine hydrochloric acid salt solution. Then, in DMF solvent containing 10 v/v % N,N-diisopropylethylamine (FUJIFILM Wako Pure Chemical Corporation), the dopamine hydrochloric acid salt solution was mixed with each of the PEG solutions (i) to (iii) so that the final concentrations of dopamine hydrochloric acid salt, PEG (i), PEG (ii), and PEG (iii) became 4 mM, 40 g/l, 80 g/l, and 160 g/l, respectively, and the mixtures were reacted by stirring at 30° C. for 3 hours. After the reaction, the solutions obtained were taken as dopamine-bound PEG solutions (i) to (iii). Dopamine binding rates were obtained by measuring the amounts of dopamine in the dopamine-bound PEG solutions (i) to (iii) diluted with a 0.1 N aqueous hydrochloric acid by using a hydrophobic chromatography system (HTEC-500, Eicom Corporation) equipped with a C18 column and an electrochemical detector according to dopamine detection conditions specified by the maker and by calculating the dopamine binding rates from the changes in amounts of dopamine before and after the reactions when the dopamine binding rates before the reactions were set to 0%. As a result, dopamine binding rates of the dopamine-bound PEG solution (i), the dopamine-bound PEG solution (ii), and the dopamine-bound PEG solution (iii) were 93%, 92%, and 90%, respectively, confirming that dopamine is bound to the PEG's sufficiently.

Subsequently, by using DMF, the dopamine-bound PEG solutions (i) to (iii) were adjusted so that the final concentration thereof became 1.5 mg/ml and light scattering particles (1) (ii) having an average particle size of 133.5 nm obtained in Example 1 was adjusted so that the final solid content became 0.5 w/v %. These were reacted and adjusted in the same manner as in Example 2 to form a 10 ml solution. In this way, light scattering particles (3) (i) to (iii) having a biocompatible polymer binding thereto were prepared.

The concentrations of light scattering particles (3) (i) to (iii) having a biocompatible polymer binding thereto were adjusted to a solid content of 0.005 w/v % by using ultrapure water, and average particle sizes were measured in the same manner as in Example 2 by cumulant analysis. As a result, the average particle sizes of light scattering particles 3 (i) (having a biocompatible polymer binding thereto, prepared by using a dopamine-bound PEG solution (i)), light scattering particles 3 (ii) (having a biocompatible polymer binding thereto, prepared by using a dopamine-bound PEG solution (ii)), and light scattering particles 3 (iii) (particles having a biocompatible polymer binding thereto, prepared by using a dopamine-bound PEG solution (iii)) were respectively 142.9 nm, 149.4 nm, and 156.4 nm. Also, PDI's (polydispersity index) were respectively (3) (i) 0.044, (3) (ii) 0.011, and (3) (iii) 0.042.

### Example 17

### Fluorescence Enhancement Effect Detected by Using Detector in UM-UC-3 Urinary Bladder Cancer Cells by Separate Administration of Fluorescent Dye, Light Scattering Particles (2) (ii), and (3) (ii)

An aqueous ALA solution (50 mmol/l) was mixed into an E-MEM medium (ATCC-formulated Eagle's Minimum Essential Medium (ATCC)) to form a 2 mmol/l ALA solution. Further, aqueous solutions of light scattering particles (2) (ii) and 3 (ii) were mixed into an E-MEM medium to respectively form a light scattering particle solution having a concentration of 0.001 w/v %.

The medium of the UM-UC-3 cells on a 6-well plate, obtained in Example 3, was removed by an aspirator, and evaluation thereof was performed under the same conditions as in Example 5 and the solution was used for detection.

Detection was performed by using an optical spectral detector (USB 2000, a small sized fiber optical spectroscope, Ocean Optics) at room temperature in a dark place. The excitation light was passed through an optical fiber (M59L01, Thorlabs) of φ=1 mm and a numerical aperture of 0.5, which is connected to a LED light source lamp (M405F1, Thorlabs) having a wavelength of 405 mm and, further, through a collimate lens (F230SMA-A, Thorlabs) having a focal distance of 4.34 mm and a numerical aperture of 0.57, and the excitation light was irradiated in a vertical direction from the upper surface on the cells on a 6-well plate. Fluorescence generated therefrom was passed through a collimate lens disposed on the upper surface in a 50° direction, the collimate lens having a focal distance of 10.9 mm and a numerical aperture of 0.25 (F220SMA-A, Thorlabs) and, further, through an optical fiber (M59L01, Thorlabs) of φ=1 mm and a numerical aperture of 0.5, and was detected by the above optical spectral detector. The excitation light output was set at 500 mA by using an LED driver (DC4100, Thorlabs). As a result of measurement of irradiation power density of the excitation light of a wavelength of 405 nm by using a light intensity measuring device (PM160, Thorlabs), the density was 10 mW/cm² at a height of the 6-well plate, the irradiation object. Setting of the optical spectral detector was performed by PC control, and a wavelength spectrum detected by the optical spectral detector was acquired by setting the exposure time, average number of measurements, and wavelength spectrum range to 100 ms, 1, and 200 nm to 800 nm, respectively. From a wavelength spectrum acquired when light was irradiated on the measurement sample, a wavelength spectrum corresponding to dark noise was subtracted and, thereafter, an intensity value at a wavelength of 635 nm, which shows a fluorescence peak, was obtained. Further, from the wavelength spectrum obtained when light was irradiated only on the cells to which, as a control, the fluorescent dye was not administered, a wavelength spectrum corresponding to dark noise was subtracted and, thereafter, an intensity value at a wavelength of 635 nm was obtained. As a reference intensity, intensity values at a wavelength of 600 nm were obtained respectively for the measurement sample and the control, and the reference intensity was obtained by subtracting the value of the control from the value of the measurement sample. Then, a value obtained by adding this reference intensity to the intensity value of the control at a wavelength of 635 nm was taken as a background intensity value in each measurement sample. And, by obtaining a difference in each measurement sample by subtracting the background intensity value from an intensity value of the measurement sample at a wavelength of 635 nm, the fluorescence detection intensity was calculated. Relative fluorescence intensity was calculated according to the following formula by using the fluorescence detection intensities obtained above:

[relative fluorescence detection intensity]=[fluorescence detection intensity under respective conditions]/[fluorescence detection intensity immediately after irradiation of light in the case of administration of fluorescent dye only].

The results were as shown in Table 13.

TABLE 13

| | | Immediately after |
|---|---|---|
| | | light irradiation |
| Relative | administration of | 1.0 |
| detected | fluorescent dye only | |
| fluorescence | separate | 4.3 |
| intensity | administration of | |
| | fluorescent dye and | |
| | light scattering | |
| | particles (2) (ii) | |
| | separate | 6.5 |
| | administration of | |
| | fluorescent dye and | |
| | light scattering | |
| | particles 3 (ii) | |

As is clear from Table 13, in the cases of separate administration of the fluorescent dye and the light scattering particles (2) (ii) and of the fluorescent dye and the light scattering particles 3 (ii), very high relative detected fluorescence intensities were obtained immediately after irradiation of light than in the case of administration of the fluorescent dye only. This is thought to be a result of intensification of scattering of visible light by the light scattering particles (2) (ii) and light scattering particles (3) (ii), administered separately from the fluorescent dye, and enhancement of fluorescence emitted from the fluorescent dye. Further, by irradiating excitation light on the cells on a 6-well plate from the upper vertical direction of the cultured tumor cells and measuring fluorescence generated therefrom from the direction of 50° above the surface, there could be observed enhancement of fluorescence emitted from the fluorescent dye. This is thought to be a result of intensification of scattering of visible light by the light scattering particles (2) (ii) and light scattering particles (3) (ii) administered separately from the fluorescent dye, despite the fact that excitation light in a visible light range was irradiated on the cells from the upper surface, and enhancement thereby of fluorescence emitted from the fluorescent dye. From the above, it became clear that, in the present invention, by using the fluorescent dye and the light scattering particles (2) (ii) and light scattering particles (3) (ii) by separate administration in a system using a detector, fluorescence is more enhanced than in the conventional case of administration of the fluorescent dye only, and the UM-UC-3 urinary bladder cancer cells can be discriminated with a higher detection intensity.

What is claimed is:

1. A method for discriminating between tumor cells and normal cells, comprising the steps of:
(a) taking a fluorescent dye having a tumor selectivity up into the tumor cells;
(b) having light scattering particles adsorbed on the surface of the tumor cells and/or uptaken into the tumor cells; and
(c) irradiating the tumor cells with light of a wavelength to generate fluorescence in the fluorescent dye at a timing when the fluorescent dye emits fluorescence in the tumor cells.

2. The method according to claim 1, wherein the step (a) is a step where the fluorescent dye having a tumor selectivity is administered in vivo to have the fluorescent dye uptaken into the tumor cells, and the step (b) is a step where the light scattering particles are administered in vivo to have the particles adsorbed on the surface of the tumor cells and/or uptaken into the tumor cells.

3. The method according to claim 1, wherein the tumor cells are epithelial tumor cells, non-invasive tumor cells, or tumor cells which constitute parenchyma of carcinoma in situ.

4. The method according to claim 1, wherein the light of a wavelength to generate fluorescence is visible light.

5. The method according to claim 1, wherein the tumor cells are discriminated by observing the fluorescence by using an endoscope and/or detecting the fluorescence by using a detector.

6. The method according to claim 1, wherein the tumor cells are those which constitute parenchyma of urinary bladder cancer, urothelial carcinoma, colon cancer, gastric cancer, esophageal cancer, cervical cancer, or biliary tract cancer.

7. The method according to claim 1, wherein an area of tumor is distinguished from a normal area by the fluorescence.

8. The method according to claim 1, wherein the fluorescent dye and the light scattering particles are not bound.

9. The method according to claim 1, wherein the fluorescent dye having a tumor selectivity includes at least one kind selected from the group consisting of 5-aminolevulinic acids and hypericins.

10. The method according to claim 1, wherein the light scattering particles include at least one kind of particle selected from the group consisting of titanium oxide, calcium phosphate, hydroxyapatite, alumina, aluminum hydroxide, silica, and polystyrene.

11. The method according to claim 10, wherein the light scattering particles have a biocompatible polymer binding to the surface thereof.

12. The method according to claim 11, wherein the biocompatible polymer is polyethylene glycol.

13. The method according to claim 11, wherein the light scattering particles further comprise on the surface thereof molecules capable of binding with the tumor cells.

14. A system for discriminating between tumor cells and normal cells comprising:
(1) a diagnostic agent comprising a fluorescent dye having a tumor selectivity; and light scattering particles, wherein the fluorescent dye and the light scattering particles are not bound;
(2) a light source which can irradiate light of a wavelength to generate fluorescence in the fluorescent dye uptaken into the tumor cells and the light scattering particles adsorbed on surface of the tumor cells and/or uptaken into the tumor cells; and
(3) an optical device for observing or detecting fluorescence generated in the tumor cells as a result of irradiation by the light source.
